(19) United States
(12) Patent Application Publication
(10) Pub. No.: US 2009/0069707 A1
(43) Pub. Date: Mar. 12, 2009

(54) METHOD TO IMPROVE NEUROFEEDBACK TRAINING USING A REINFORCEMENT SYSTEM OF COMPUTERIZED GAME-LIKE COGNITIVE OR ENTERTAINMENT-BASED TRAINING ACTIVITIES

(76) Inventor: Joseph A. Sandford, Richmond, VA (US)

Correspondence Address: Thomas & Raring, P.C., 536 GRANITE AVENUE, RICHMOND, VA, 23226, US

(21) Appl. No.: 12/206,083

(22) Filed: Sep. 8, 2008

Related U.S. Application Data
(60) Provisional application No. 60/970,389, filed on Sep. 6, 2007.

(51) Int. Cl. A61B 5/0482

(52) U.S. Cl. 600/545; 600/544

(57) ABSTRACT

This invention consists of a neurofeedback training system that utilizes EEG activity based goals to help motivate trainees to learn to produce and sustain desirable EEG activity by enabling them to earn “game play” time. The method provides positive behavioral reinforcement to trainees by rewarding them with the opportunity to begin or continue to participate in computer game-like or entertainment-based training activities (e.g., video games, educational games, movies, music or computerized cognitive training exercises) whenever the specified EEG activity goals are achieved and maintained for a set time period. The method also provides, in one embodiment, negative reinforcement in the form of the loss of game play or entertainment opportunities whenever the specified EEG activity goals are not achieved or maintained for a specified time interval. The subject method provides simultaneous real-time, concurrent visual, auditory and/or quantitative informational feedback during the game-like computer play or entertainment that puts the trainees “in charge” so that they can win or lose game play time based on their ability to produce the specified brainwave activity that is being reinforced. The purpose of this method is to help trainees learn to maintain desirable, alert mental states associated with successful cognitive and emotional functioning during neurofeedback training exercises and during any life activity requiring active mental engagement.

This application claims the benefit of provisional application No. 60/970,389 filed on Sep. 6, 2007, which is incorporated by reference herein.

### FIELD OF THE INVENTION

This disclosure relates to a method to more effectively train and motivate a person's control of electroencephalographic (EEG) activity through operant conditioning and goal achievement feedback provided in the provision of neurofeedback training. More specifically, the method disclosed herein involves the presentation of real-time and concurrent feedback of a person's success or failure in achieving an EEG activity goal indicative of a desirable mental or emotional state while he or she is simultaneously engaged in viewing or interacting with any type of visual or auditory display that requires or stimulates mental activity necessary in order to process, respond, engage or understand.

### BACKGROUND

In general, neurofeedback is best understood as a operant conditioning training process that involves measuring a person's brainwave activity and then communicating specific information to him or her in real-time so that an individual can become more aware of the psychophysiological processes of one or more selected brain areas. The purpose of neurofeedback training is to enable individuals to learn how to gain conscious control of specific brainwave frequency patterns and/or change the interaction and communication between the different functional centers of their brain. This increase in a person's control of their brainwave functioning has been found in a large number of scientific studies to lead to improvements for many individuals in respect to their self-regulation or in the reduction of symptoms that negatively impact their quality of life. An annotated bibliography of most of these research studies is available online at www.isnr.org.

As an example, some young people diagnosed with Attention-Deficit/Hyperactivity Disorders (ADD/ADHD) who were described by their parents and teachers as being hyperactive have been found in a number of scientific studies to significantly reduce their hyperactive behavior after neurofeedback training. In many clinical treatment cases, neurofeedback training has typically involved increasing the brainwave frequency defined as the Sensorimotor Rhythm (SMR) in the central area of the brain called the primary motor cortex. Based on behavioral and operant learning theory, the two essential factors that are required in order for neurofeedback training to succeed is that the individual being trained needs both 1) accurate information about when the targeted brainwave activity is and is not manifesting and 2) a measure of its signal strength (e.g., its amplitude in microvolts), coherence (i.e., relationship to other signals in different brain sites), or other different types of mathematical measures of signal activity (e.g., increased signal amplitude for a specified EEG frequency bandwidth in one brain region and a simultaneous decreased signal amplitude for a specified EEG frequency bandwidth in different brain region).

The commonly used training methods of neurofeedback are often perceived by the participants in the training process as boring, repetitive and difficult to master. The typical training method require upwards of 40 or more sessions of feedback training. The trainee also may become easily frustrated and lose motivation during initial training sessions because the process for learning how to control brainwave activity cannot easily be communicated via verbal instructions. In other words, the trainer cannot tell the trainee how to produce the desired EEG activity as the learned behavior is non-verbal and must be experientially learned through informational feedback.

The principle behind neurofeedback is similar to a “mind mirror” in that it is used to help a person view their mental activity better so that they can learn through practice and experience how to control it whether such activity involves increasing or decreasing specified EEG activity in a designated area of their brain. Typically, most trainees voice confusion when neurofeedback training begins and state that they do not know what it is that they are supposed to be doing to achieve control of their EEG activity. Patients diagnosed with attention deficits are particularly vulnerable to these feelings of frustration. Due to these feelings of a lack of control or frustration that often occur due to the inherent requirements of the learning task, it is clear that many trainees, particularly those with ADHD, may lose motivation and/or confidence in their ability to achieve the potential benefits of neurofeedback. Further still, negative feelings may also then manifest that impair their ability to achieve the goals of neurofeedback training due to low frustration tolerance and their possible difficulties in persevering until observable progress can be made. Thus, the achievement of neurofeedback training goals often takes several months and is often a very challenging ability for trainees to learn. The trainers can also be challenged by the difficulty of the process and the skills for each trainer will vary so that inconsistent results may be obtained for independent trainees.

The first neurofeedback training machines used only illuminated lights and simple numeric counters to signal whenever the desired EEG activity was detected. Audio feedback was later added so that trainees could learn under ‘eyes-closed’ conditions to produce alpha brainwaves (8-12 Hz) which were naturally more prevalent when a person's eyes were closed. In 1991, one of the major pioneers in the field of neurofeedback, Joel Lubar, designed and helped a company called Autogenics build one of the first computerized neurofeedback training systems. This system utilized animated computer graphics as part of a simple video game to communicate to the trainee whenever the targeted EEG signal was being produced. For example, a fish would swim around in a maze towards a goal and the speed that the fish swam was controlled by the EEG activity of the trainee. The purpose of using visual and audio feedback was to make the neurofeedback training more interesting and game-like. This general model for training is used today in almost all neurofeedback training systems. The informational and motivating value of this type of feedback is better than just simple light and numerical counting displays, but it is limited in that the trainee is only given a feedback signal that the desired EEG activity was momentarily produced and the simplistic nature of the video game is not very engaging, as the trainee's only means for game interaction is by controlling their EEG activity. The use of computer displayed animated feedback in this simple, mentally controlled feedback format provides only limited cognitive stimulation, and very minimal entertainment value, as this method does not provide trainees any opportunity to more easily physically interact via a game input controller (i.e., a joystick or gamepad) in the types of video games that are more challenging and motivating.

In order to help generalize the effects of neurofeedback training and to engage the trainee in purposeful mental activities that stimulate appropriate increases in cognitive functioning, Joel Lubar would monitor EEG activity while trainees were performing academic tasks, such as reading, writing or solving arithmetic problems. He would then concurrently provide the trainees with auditory neurofeedback and/or verbal summaries of their neurofeedback progress while they were engaged in the assigned academic task. This method of neurofeedback training provided trainees solely with limited audio feedback regarding the achievement of neurofeedback goals that would be difficult to concurrently process given the type of academic tasks selected that required internal verbalization in order to perform them. This training methodology was inherently limited. For instance, it did not provide trainees with visual and/or quantitative feedback that they could concurrently process on their own

The possible value of a training protocol that combined neurofeedback with cognitive training has been explored in my earlier work (Sandford, 1994). This protocol required two computers, one for neurofeedback training and another one that concurrently provided cognitive training exercises for trainees. Later, Tinius and Tinius in 2005 published their research combining neurofeedback and cognitive training approaches using this two computer approach that was partly based on my ideas and used some of the cognitive training software I had developed.

One computer was used to provide feedback of the desired EEG activity and participants performed cognitive game-like exercises on a second computer. The two computers operated separately, and the neurofeedback training results had no effect on the cognitive training tasks. Tinius' research found that adults who had been diagnosed as having either mild traumatic brain injury or ADHD improved in their attention abilities and psychological functioning after only 20 sessions of this combined training approach. Tinius' training method required the use of two computers and relied on a neurofeedback system that primarily used auditory tones and trainer verbal coaching to signal to trainees when the desired brainwave state was present. At the same time using a second computer, these subjects were concurrently required to perform visual and auditory computerized cognitive tasks that trained attention, mental processing speed and overall accuracy. For these cognitive training exercises both visual and auditory feedback, as well as, a quantitative score and trainer coaching reflecting accuracy were provided while trainees listened to the tones emanating from the neurofeedback computer that signaled either the presence or absence of an alert mental state.

Using this two computer approach, it was not possible for trainees to easily view visual or quantitative feedback of their brainwave activity without shifting focus away from the cognitive exercise that they were doing without distracting themselves and, thus, disrupting and, in all likelihood, impairing their performance. While it was technically possible to completely stop the cognitive training exercises and have the trainees focus solely on the visual and auditory neurofeedback information if they were not achieving the EEG activity goals, this was never implemented in this study and would have been disruptive as the cognitive training exercises used in this research would have had to been restarted from the beginning resulting in the loss of this training data. Also, the concept of clearly communicated EEG activity training goals for the trainee that are used in a systematic way with preset quantified criterion to provide both positive and negative reinforcement that controls the pausing and restarting of motivational game-like, cognitive or entertainment activities as disclosed in this subject method is new to the field of neurofeedback training. Thus, the feedback provided to trainees of their EEG activity was limited primarily to brief auditory tones that communicated momentary goal success and their success or failure in respect to any desired EEG activity goals was not used to provide either positive or negative motivational reinforcement.

The specific auditory feedback of brainwave activity used in this two computer training method was simultaneous and continuous during the cognitive training and the training protocol did not result in any stopping of the cognitive training exercises for any reason. Trainees were instructed to focus primarily on the cognitive training exercises and secondarily verbally informed of any improvements observed in their EEG activity. Since the trainees' EEG performance did not effect or change in any manner the cognitive game-like exercises, their success in controlling their EEG activity had no specific consequential motivational value for them. Also, this method did not provide any opportunities for subjects to focus and receive only neurofeedback training without the requirement to also have to simultaneously perform a cognitive task making the task of learning to control brainwave activity possibly very challenging for some trainees.

These researchers concluded that it seemed possible that some trainees benefited more from the neurofeedback, others from the cognitive training, and some from the combination of both methods. Thus, this research supported the value of combining neurofeedback and computerized cognitive game-like training as beneficial in improving attentional and psychological functioning.

As noted above, the original neurofeedback computerized system developed by Dr. Lubar relied on real-time, clearly discernible visual and auditory feedback in order to help trainees learn to control their EEG activity. However, viewing and trying to understand how to produce and control EEG activity using solely an operant conditioning method is recognized in the field as inherently boring and initially very challenging. Consequently, several methods have been developed to help increase motivation and make the challenging and somewhat boring task of learning EEG biofeedback inherently more fun. The concept and ideas underlying these approaches to make the neurofeedback learning task more fun focuses primarily on using a “difficulty adjusting” method to communicate in various direct and indirect ways a person's success at controlling the desired EEG activity. These difficulty adjustment methods are implemented in different ways to modify physically interactive video games that typically require the use of a hand-held game controller in an attempt to motivate trainees to learn EEG control. The use of video games as a feedback modality was selected as they are commonly perceived as inherently motivating and fun by most children, adolescents and many adults, as well.

The value of using an interactive video game modality as a motivational factor that can be used to enhance neurofeedback training was researched by NASA. The first method developed by Pope (1994) used a difficulty adjuster that made the video game activity easier whenever the trainee achieved the desired goal of producing EEG activity associated with being more attentive and, likewise, the game demands became harder if the trainee was not producing the desired EEG activity. In other words, trainees could more easily win when they paid attention as measured by their EEG activity and, if they were not able to pay attention well, then the game play was harder and, thus, they were more likely to lose. For example, if the purpose of the video game was to shoot at targets that were moving using a game controller and the trainee was able to produce the desired EEG activity, then the targets would move slower and, hence, be easier to hit. In this example, whenever the trainee did not produce the desired brainwave activity the targets would speed up and be harder to hit. The trainee was also provided a symbol that represented their level of attentional functioning based on their EEG activity. This method was designed to indirectly communicate to the trainee based on the presentation of video game objects when they were paying attention or not. It does have the possible inherent drawback of making the video game play too easy when the trainee is paying attention well and, in this case, the video game play could possibly become boring, which would then lower a trainee's motivation. In fact, this method could also possibly result in a trainee becoming overwhelmed and frustrated when game play difficulty is increased because they are not able to pay attention and, as a result, continue to repeatedly lose the video game. This method may also provide possibly confusing feedback to the trainee as to whether they are maintaining and succeeding in controlling their EEG activity since no quantification is provided in respect to scores that the trainee can use to easily measure their success. Thus, the success and motivational value of this method is likely to be variable in its enhancement of neurofeedback training.

In another NASA method developed by Palsson (2002), a different method was used to increase and decrease the difficulty level of a video game play based on EEG activity by impairing the trainee's ability to control video game activity. As an example of this method, the game input device used to control game play for a PlayStation video game console is temporarily disabled or impaired whenever the trainee loses their attention. This disruption of the game controller makes it more difficult for the trainee to move their game character or to respond quickly to game elements. This difficulty adjuster was designed to punish the trainee for the failure to control their EEG activity by causing them to perform poorly in the video game and in this negative way motivate them. Thus, failure to control their EEG would often lead to losing the video game for trainees. In some applications of this method a display system was also used to communicate visually a value related to the trainee's attentional state, as is commonly done in other neurofeedback training systems. However, this method did not utilize quantitative or auditory feedback modalities or clearly communicate any threshold of success that the trainee could use to measure their progress. While the desired EEG activity is communicated to the trainee in real-time using this method both directly and indirectly as described above, the difficulty adjuster feedback system used could easily lead to frustration and feelings of failure. These negative emotions could easily become overwhelming as the trainee struggles and fails to control their EEG, which results in their game controller malfunctioning and then he or she winds up losing the video game. In this method an increase in motivation to perform successfully in the control of EEG activity is attempted with the reward of playing an entertaining commercial video game, but any lapse or decrease in attentional control which is likely to frequently occur, particularly at the beginning of training, is then punished. Thus, the motivational value of this method may quickly be lost, because of a trainee's negative emotional reactions to this method of training that relies on punishment.

One other similar version of the difficulty adjusting method was developed by Freer (2003). His method is based on the modulation of the pace of the stimuli of video games that are designed to train mental processes such as short-term memory sequencing, visual discriminatory processing, visual tracking and auditory discriminatory processing. In this method, when the trainee is producing EEG activity that is above a threshold level, they are then considered more focused, attentive and “on-task.” The pace of the presentation of the stimuli in these cognitive training video games is controlled by whether or not the trainee is on-task. For example, stimuli such as different colored asteroids will appear only when the trainee is on-task in order to train visual discrimination abilities. Once the asteroids appear the trainee has to press the space bar, if the asteroid is one color (e.g., red), and not press the space bar, if it is a different color (e.g., green). For the game stimuli to continue to be presented, the trainee must produce EEG activity above the threshold level indicative of focused attention. Thus, whether the trainee is on-task is communicated in only an indirect manner to the trainee as the game stimuli is presented more frequently whenever they are focused. This method motivates trainees to learn to focus more because doing so makes it possible for them to score more points in the game. No quantification of EEG activity is provided in respect to scores that the trainee can use to measure their success for this method. In those cases when the pace of the game stimuli presentation is slower in response to the trainee's failure to maintain their attention to the task, some trainees may prefer this less demanding task and, thus, their motivation to produce the desired alert mental state may lessen or they may incorrectly believe that they are actually succeeding when they are not because their accuracy becomes higher. This difficulty adjusting method does not provide any quantitative or auditory information to help the trainee learn when the desired EEG activity is being produced or not. In addition, the visual feedback of the presence or absence of desired EEG activity reflected as it is in the variable pace of the video game has to be first recognized and then correctly interpreted by the trainee leading to the possibility of easy misinterpretation and, thus, impairing learning of EEG control.

A somewhat similar difficulty adjusting method to that used by Pope (1994) for communicating the success or failure of a trainee's control of EEG activity has been proposed by Collura (2007) as a way to improve motivation during neurofeedback. In this method, the difficulty level of the action and content of the video game and the intelligent decision making behavior of an embedded game player who represents the trainee in a video game format, such as basketball, is used to convey the success or failure of trainees in respect to their control of designated EEG activity. In this method, the trainee may become more motivated by the game-like quality of play if the video animation and game activity is of the same high quality as is available in commercial video games. This method has the embedded game player making essentially good or bad decisions, being able to move more quickly, and having exposure to opportunities or help in a video game world based on the success of the trainee in producing the desired EEG activity. Like Pope's method, this approach adjusts the game difficulty based on whether or not the trainee produces the desired EEG activity indicative of focused attention making the game easier or harder to win. The game difficulty of this method and the possibility of “good” decisions made by the embedded game player would be increased or decreased based on the trainee's attentional mental state as measured by his or her brainwave activity. The informational feedback of this method is by design inherently indirect and, as a result, trainees may possibly become confused as to whether they are maintaining and succeeding in controlling their EEG activity.

The lack of the provision of a measure clearly quantifying the real-time EEG analysis in this difficulty adjusting method taught by Collura (2007) would make it difficult for a trainee to accurately measure their success. Given the way feedback is presented in this method, in some cases, when the game play became easier due to the trainee's success at controlling their EEG activity, the actual reward, such as a door opening, would likely occur sometime later “down the hall” when such game activity is relevant in the game environment. Thus, in this example positive feedback in the game activity may occur at a time when the trainee is concurrently producing a non-desirable EEG state (i.e., when the door actually opens later down the hall) significantly impairing learning since neurofeedback control is based on operant conditioning principles that require that the reward be closely paired in time to the desired behavior. This method also does not stop or interrupt the game play at any time in order to clearly communicate failure, nor does it provide real-time and discrete visual, quantitative or auditory informational feedback to help the trainee learn whether the desired EEG activity is being produced or not. Thus, the motivational value of this method is in its similarity to commercial video games.

While participating in neurofeedback training and viewing video game play may possibly be entertaining, like a movie or TV show, it is not likely to provide the relevant specific, detailed, or timely information that is necessary in order to train the success or failure of a person's control of their brainwave activity. Given the complexity and sophisticated nature of the feedback provided in this method via the adjustment of the game difficulty involving the action and content of the game and the embedded game character's good or bad decisions in respect to various video game goals, trainees may also easily become confused and misinterpret how their ability to control their EEG activity is related to their game success. Known techniques do not provide the trainees with any substantive way to earn or lose some type of immediate reward related to the performance of neurofeedback goals that was under their control.

### SUMMARY OF THE INVENTION

This subject method involves the use of visual, auditory or quantified informational feedback of a trainee's real-time EEG activity and goal-oriented success while he or she is simultaneously engaged in viewing or interacting with any type of visual or auditory display that requires or stimulates mental activity necessary in order to process, respond, engage or understand. The achievement of the EEG goal activity is used to control whether the concurrent video or auditory activity is either initially provided or is continued to be provided. This method provides both positive and negative reinforcement in order to motivate and help a trainee learn how to achieve desirable EEG goals by discontinuing or not providing a pleasurable or stimulating activity such as a video game, computerized cognitive training, music, educational game or movie when the goal is not achieved and then requiring them to focus solely on neurofeedback training in order to better learn to achieve the EEG goal and then “win” the opportunity to participate in the stimulating and motivating video or auditory activity.

The EEG real-time goal-oriented informational feedback is presented using the same computer or electronic equipment that provides the visual or auditory display. In this method, the EEG analysis and feedback components are a separate part of the system and, thus, can be used to start and stop an independently running game-like or entertainment activity such as a video game, movie, music, educational game or computerized cognitive exercise depending or whether or not the desired EEG activity goal is achieved. Unlike known techniques, it does not change the difficulty level or pace of the game-like or entertainment activity used to help motivate trainees. Nor does it use the trainee's EEG activity to provide neurofeedback feedback training within the game-like activity or to influence a person's success or failure in any way in the game.

In one embodiment, the subject method provides trainees with real-time, visual, auditory and/or quantitative goal-oriented informational EEG feedback using only one computer or electronic device. The computer system or electronic device used (e.g., TV, digital video recorder, hand-held or component video game device, iPod, CD player, DVD player or any other type of game-like or entertainment electronic device) displays or presents simultaneously on the same video screen, a connected external primary or secondary video display or through the same audio system both the EEG informational feedback and the game-like or entertainment activity. Thus, trainees do not need to be distracted and shift their attention in order to look at a second computer screen or other type of electronic display device used for EEG analysis, feedback and display while they are engaged in the learning process of this method. Also, they do not need to rely on a trainer to summarize and verbally inform them of their EEG goal score processed and visible only on a separate second computer or electronic device. This method also incorporates both a positive and negative goal-oriented reinforcement system that has not previously been recognized or implemented, and it does not rely or require any trainer verbal feedback or interpretation.

This subject method is based on a trainee's ability to achieve for a specified time period any type of desirable EEG training goal. As noted in the above, a person's ability to learn to control their EEG activity is often initially difficult and takes considerable training time. In the subject method, a clearly understandable EEG goal is presented that the trainee must either attain or maintain in order to “win” the opportunity to engage in a separate game-like or entertainment activity. It puts the locus of control for the continuation or discontinuation of the game-like or entertainment reinforcement clearly “in the minds” of the trainees. Also, unlike prior art, in this method the operant conditioning required for successful neurofeedback regarding the occurrence of desired EEG activity is clearly and continuously communicated in real-time and summarized in respect to whether a goal that is meaningful to the trainees is being achieved or not. One advantage of this method, which significantly distinguishes it from prior art, is that it clearly helps trainees to feel in control and to be more motivated to learn control, as the positive and negative consequences are always readily apparent and not indirectly communicated in the level of game-like difficulty, embedded game player decision making that influences game outcome or the pace of game stimulus presentation and activity.

In the subject method, trainees clearly know whether they are achieving the desired EEG goal and, if not, the game-like or entertainment activity is temporarily interrupted. For example, one type of EEG activity goal that can be used is the percent of time (e.g., 70%) that a trainee's EEG amplitude measurement is above a threshold level for an EEG frequency bandwidth that is indicative of ‘paying better attention’. In this case, when trainees are determined to not be adequately paying attention, then an interruption of the motivational fun activity occurs and the trainees are then provided only neurofeedback training in order to help them learn how to produce the desired EEG goal activity. Once they are successful in achieving the EEG activity goal (or the specified neurofeedback training exercise time expires), the trainees are positively reinforced by allowing them to return to the game-like or entertainment activity. Thus, whenever trainees are not successfully demonstrating control of the desired EEG activity, they are then provided specific, brief neurofeedback only training to help them more easily learn control of their EEG. Once the trainees successfully demonstrated the achievement of desirable EEG activity goals, then neurofeedback training continues while they are simultaneously required to be mentally active in performing an independent cognitive, stimulating or enjoyable activity.

In another preferred embodiment, the subject method provides an alternative starting procedure in which trainees first have to “earn” the game-like or entertainment play time by producing the desired EEG activity goal and then if this goal is not maintained, they lose their “play” time. This variation of requiring trainees to earn game-like or entertainment activity by achieving the goal of producing desirable EEG activity enables this method to be used to provide only positive reinforcement. For example, in this alternative starting procedure an adolescent aged trainee would be required to successfully achieve a specified EEG activity goal for a fifteen minute training period before the electronic device switches and turns on the TV. In this case, the trainee could then stop the neurofeedback training and enjoy a half-hour TV show. The computer or electronic device could also record and store TV or video game “credits” based on the trainee's success in achieving EEG goal directed activities and they can be “cashed in” at a later time for play time.

One major benefit of this method is that it reduces the problem of boredom, frustration and confusion that can occur during traditional neurofeedback training methods typically used in most neurofeedback systems today. These negative emotional states can impair the learning process. With this method the trainee can be queried as to what type of activities they enjoy and those activities, such as watching their favorite movie, used to help motivate them to learn how to control and maintain desirable EEG brainwave patterns associated with positive mental and emotional functioning.

In addition, this method provides the opportunity for trainees to learn to produce and maintain EEG patterns not just during neurofeedback training, but also in more real life situations, such as when they are reading and/or studying for tests. Thus, this method can easily be used to promote the generalization of neurofeedback training by helping trainees to learn to maintain EEG activity goals associated with more active mental processing when they are performing academic or work place types of tasks on the computer, such as word processing.

The specific EEG activity goals can be either simple or complex in this method. The achievement of any EEG activity goal in this method can be presented in both immediate and goal criterion format. Immediate visual, auditory or numeric value static or animated feedback can be continuously displayed or its presentation can be modified to suit the preferences of the trainee. Also, success or failure based on the maintenance or production of the desired EEG goal activity can be communicated in quantified numeric feedback using percent of success or any other type of scoring system that symbolically communicates in numerical, visual and/or auditory modalities success or failure of desired EEG goal activities.

Any neurofeedback training protocol currently used in clinical treatment centers or in research laboratories can be modified by the subject method to include set EEG activity goals. The types of neurofeedback training feedback signals or analysis used in creating these treatment goals can be either simple or complex and can be based on the EEG and/or other measureable psychophysiological modalities alone (e.g., heart rate, external skin temperature or EMG activity) or in combination. EEG normative or raw measures of amplitude, phase, coherence, co-modulation, Loreta EEG analysis, QEEG z-scores, inter or intra-hemisphere differences or almost any other type of EEG activity that can be quantified can be used with this subject method to set specific EEG training goals. These goals can be based on any of the above measures from one or more of the 10-20 brain sites either singly or in any type of combination and calculated using additive, summation, threshold based, standard deviation or other more complex algorithmic mathematical formulas.

Overall, this invention consists of a neurofeedback training system that utilizes EEG activity based goals to help motivate trainees to learn to produce and sustain desirable EEG activity by enabling them to earn in a variety of ways “fun” time. The method provides positive behavioral reinforcement to trainees by rewarding them with the opportunity to begin or continue to participate in computer game-like or entertainment-based training activities (e.g., video games, educational games, movies, music or computerized cognitive training exercises) whenever the specified EEG activity goals are achieved and maintained for a set time period. The method may also provide negative reinforcement in the form of the loss of game play or entertainment opportunities whenever the specified EEG activity goals are not achieved or maintained for a specified time interval. In this way, the subject method helps to generalize a trainee's ability to learn to maintain desired EEG activity while simultaneously performing relevant mental skill building that are meaningful in improving emotional stability and functional performance in a variety of recreational and vocational life activities. These EEG activity goals can be based on either simple or complex neurofeedback training protocols using one or more brain sites and any type of raw, normative or algorithmically derived measure of brainwave activity. The EEG feedback goal activity can also be combined with training goals based on any other psychophysiological measurement (e.g., heart rate, external skin temperature or EMG activity). The subject method used provides simultaneous real-time, concurrent visual, auditory and/or quantitative informational feedback during the game-like computer play or entertainment that puts the trainees “in charge” so that they can win or lose game play time based on their ability to produce the specified brainwave activity that is being reinforced. The purpose of this method is to help trainees learn to maintain desirable, alert mental states associated with successful cognitive and emotional functioning during neurofeedback training exercises and during any life activity requiring active mental engagement.

### BRIEF DESCRIPTION OF THE FIGURES

The foregoing, and additional objects, features, and advantages of the present invention will become apparent to those of skill in the art from the following detailed description of a preferred embodiment thereof, taken in conjunction with the accompanying drawings, in which:

FIG. 1 illustrates a flowchart of one embodiment of this neurofeedback training and reinforcement method for improving the training and learning of desirable EEG activity goals disclosed herein. The method in FIG. 1 describes beginning with EEG activity goal training simultaneously provided with a motivational fun activity.

FIG. 2 illustrates a flowchart that describes an alternative feedback training method of this invention that begins with requiring the achievement of the EEG activity goals and then the successful achievement of them results in “earning” time to engage in a motivational fun activity.

### DETAILED DESCRIPTION

Various feedback models can be used with this subject method to provide the positive reinforcement for the trainee of being able to engage in a motivational activity that is desirable because it is fun, entertaining or mentally stimulating. Also, this subject method provides for the presentation of relevant EEG goal oriented feedback that has designated criterion values to be achieved and maintained while the trainee is concurrently engaging in the game-like or entertainment activity.

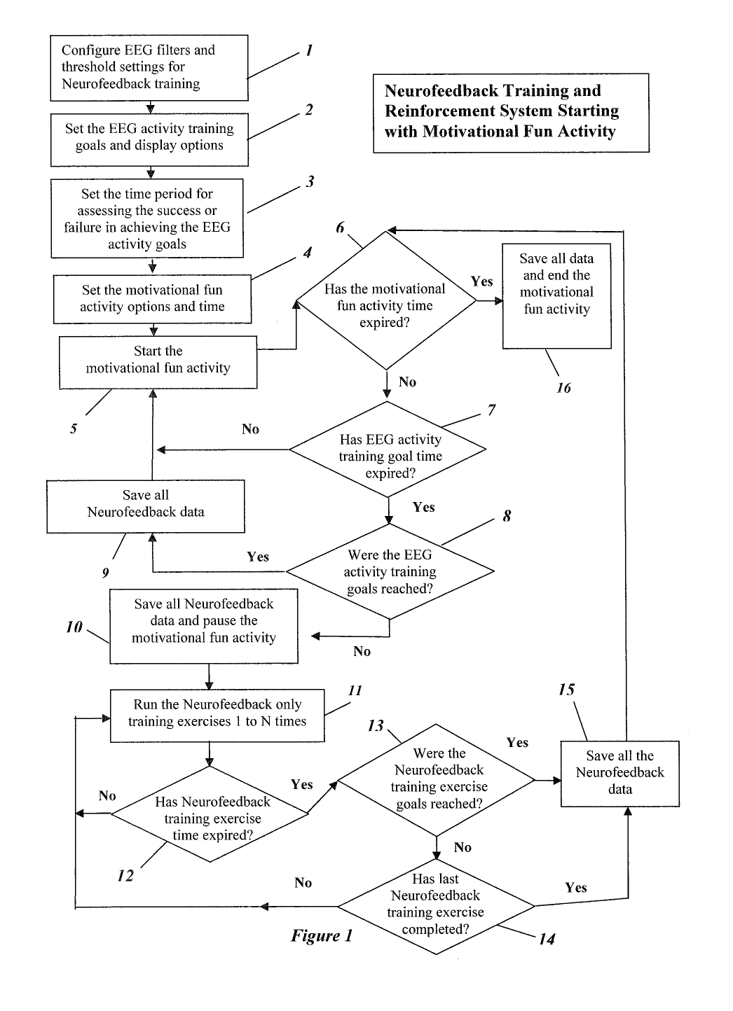

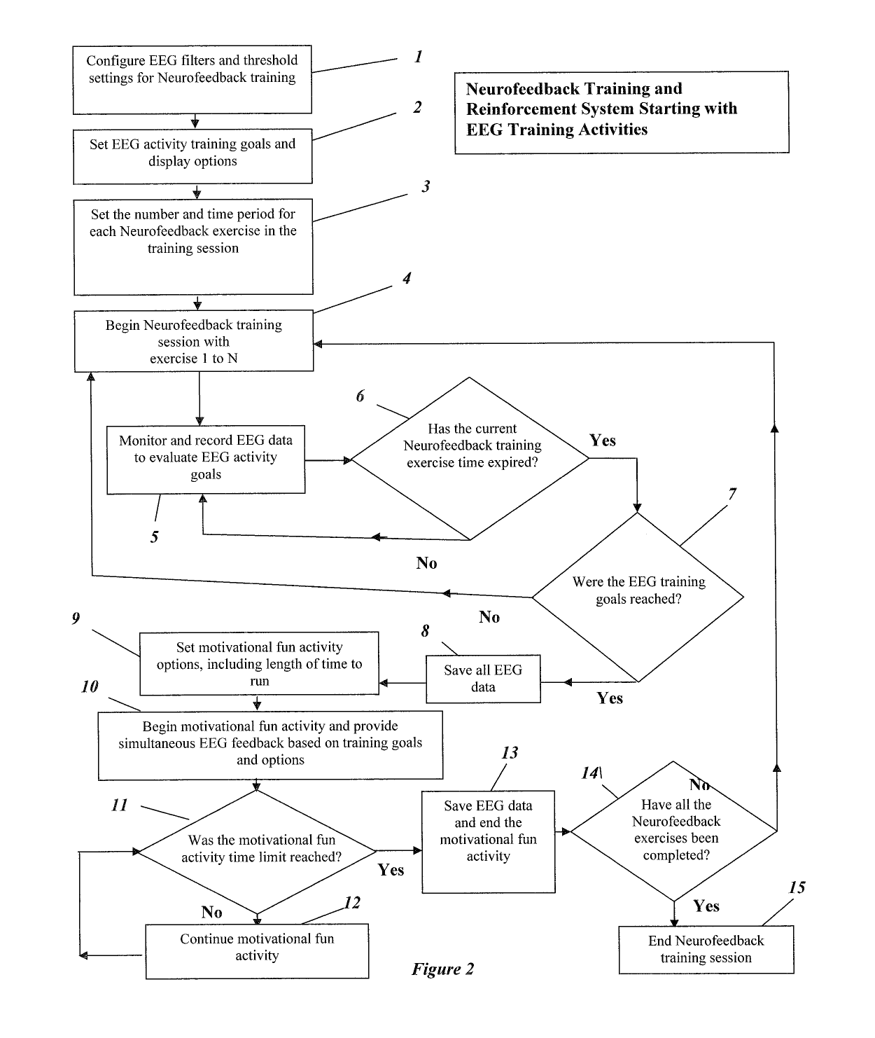

Turning now to FIGS. 1 and 2, there is illustrated in FIG. 1 an embodiment of this method that provides the trainee continuous EEG feedback information and utilizes a training paradigm that stops the motivational fun activity whenever the trainee fails to maintain the EEG activity goal for a specified time period. In this case, if the trainee is able to maintain the desirable EEG activity goal during the designated time period, then he or she does not lose the opportunity to continue to engage in the motivational fun activity. However, if the trainee fails to achieve the criterion used in assessing the EEG activity goal, then they do lose the privilege of being able to continue the game-like or entertainment activity, which constitutes negative reinforcement. The trainee is then provided only neurofeedback training exercises in order to help them learn how to better control the desirable EEG goal activity. Once trainees succeed then they “earn” back play time and the motivation fun activity resumes where it left off (i.e., are positively reinforced). The EEG goal oriented feedback also continues to be provided and progress is repeatedly assessed as scheduled. Unless the trainee maintains the desirable EEG activity goal, they again lose access to the game-like or entertainment activity and the above training process repeats itself again until the preset time period of training is reached.

With reference to the flowchart of FIG. 1, the subject method requires the configuration of the EEG filters and settings related to specifications pertaining to the EEG activity goal reinforcement. This step is identified as element 1 of FIG. 1. In this disclosed method, the EEG activity training goals are set along with display options that may include visual, auditory or quantified numerical feedback information 2. The time periods for assessing the success or failure of the EEG activity goals are then configured 3. Next, the overall training time period and any options related to various aspects of the motivational fun activity are specified 4. The motivational fun activity in any embodiment of the subject invention can by any known entertainment activity. The motivational fun activity or entertainment activity is then started 5. A check is continuously made during game play as to whether the training time is up 6 and, if not, then EEG activity training goal is evaluated as to whether the time period to start assessment has expired 7. In the case where the motivational fun activity training time has expired 6 then all data is saved and the training is ended 16. Otherwise, as long as the time required to assess the achievement of the EEG activity goals has not expired 7, then the training continues 5.

In the case where the EEG activity training time has expired 7, then a test is made 8 in order to see if the EEG training goals were reached. If these goals were achieved, then all the neurofeedback data is saved 9 and the motivational fun activity continues 5. If the EEG activity training goals were not met, then the neurofeedback data is saved and the motivational fun activity is paused 10. The neurofeedback training exercises are then presented separately for the designated number of times 11. Each instance of a neurofeedback training exercise is tested 12 until the training time period has expired and then the EEG activity goals are tested to see if they have been reached 13 and, if so, all the neurofeedback data is saved 15 and a test is then made as to whether or not the motivational fun activity training time has expired or not 6.

In the case when the neurofeedback training goal for a specific segment of training did not result in goal attainment, then a test is made as to whether all the specified neurofeedback training exercises have been completed 14. If no, then neurofeedback training continues 12 until either the EEG activity goals are reached 13 or the last neurofeedback training exercise is completed 14. Then all the neurofeedback data is saved 15 and a test is then made as to whether training is complete or not 6. Training ends if it is completed 16 and, if not, continues as specified in detail above.

In FIG. 2 an alternative embodiment of this method is illustrated that first requires the trainee to successfully produce the EEG activity and “earn” the opportunity to engage in a fun, mentally stimulating, or entertaining motivating activity. Initially, the trainee is provided solely EEG feedback information and is periodically assessed as to whether the specified EEG activity goal is achieved. In this case where the goal is achieved then the trainee is permitted access to the motivational fun activity for a specified time period (i. e., positive reinforcement). EEG informational feedback is always provided during the time that the trainee is engaged in the game-like or entertainment activity, but in this embodiment of the subject method the trainee is allowed to continue participating in the motivational fun activity until the specified “play” time has expired. The trainee may be then given additional chances to earn more play time by successfully achieving the EEG activity goals, if more neurofeedback training sessions are set in the training plan options. In this case, only the EEG goal oriented feedback is provided until the designated goals are achieved and then the trainee is again provided access for a fixed time period to the motivational fun activity. Otherwise, training simply ceases. This embodiment is applicable in cases where TV or Internet time is the motivational fun activity. In this case, the trainee may have to first work with neurofeedback training for 15 to 20 minutes and demonstrate control of their EEG activity by achieving the specified goal in order to be rewarded with opportunity to watch their favorite TV program or browse on the Internet.

With reference to the flowchart of FIG. 2, the subject method requires the configuration of the EEG filters and settings related to specifications pertaining to the EEG activity goal reinforcement. This step is identified as element 1 of FIG. 2. In this disclosed method, the EEG activity training goals are set along with display options that may include visual, auditory or quantified numerical feedback information 2. The time periods for assessing the success or failure of the EEG activity goals are then configured 3. Next, the neurofeedback training sessions are started 4. The EEG activity is recorded and the success or failure in achieving them is continuously communicated to the trainee 5. A check is continuously made during game play as to whether the neurofeedback training time is up 6 and, if not, then the neurofeedback training continues 5. In the case where the neurofeedback training time has not expired 6 then a test is to determine if the EEG training goals were reached 7. If not, then the next neurofeedback training session is started 4. In the case when the training is successful the EEG data is saved 8. The options and time period are then set for the motivational fun activity 9 and then this motivational fun activity begins with concurrent Neurofeedback training 10. The motivational fun time training limit is then tested 11 and this activity continues unless the time is up 12. When the motivational fun activity time has expired then the EEG data is saved and the motivational fun activity ends 13. A test is then made to see if all of the neurofeedback training exercises have been completed 14. If they have all been completed, then the neurofeedback training session ends 15. Otherwise, the next neurofeedback training session is started 4.

Other more complex and sophisticated training goals and options can also be developed to provide reinforcement for the attainment of EEG activity control using this subject method. This subject method can be easily modified to evaluate and train a wide variety of different measures and aspects of brainwave activity based one or more goals being achieved. For example, the reinforcement of coherence for different brainwave sites or amplitude of EEG activity reflecting overall brain functioning using normative z-score, multi-site qEEG or fMRI data can be used in creating the EEG activity goals. Also, the time that goals have to be maintained or sustained during the assessment period can be varied. Goal settings and criterion can also be modified for any number of EEG channels; any EEG bandpass filter range or have their threshold settings automatically adjusted. Additionally, threshold goals could be configured for minimum or maximum time periods above or below threshold levels. These levels can be based on the ratio of the amplitude of one EEG filter bandpass to another or the minimum number of bursts of activity where a burst of activity is defined as the continuous occurrence of designated brainwave activity at a predefined level for a specified period of time. An increase or decrease of a trainee's brainwave activity by a percentage level of the trainee's baseline standard deviation could also be used as a success goal in this system.

Positive and negative reinforcement in this subject method can be modified in many ways. For example, the neurofeedback training exercise could provide a “virtual game tokens” that can be redeemed for different lengths of time that the trainee is allowed to play the motivational fun activities. Also, tokens could be used to obtain the privilege to choose what type of motivational activity the trainee is permitted to play. The tokens could then be redeemed for additional playtime on different game machines in the same way that an arcade token is used in a video arcade. This system could also be used to control the video game time for commercial video game machines or to control access to TV time. Thus, the more successful that the trainee is in achieving the neurofeedback training goals, the longer the trainee would be permitted to engage in the available rewarding activities.

The invention is not limited to a specific number of EEG channels, filters, types of EEG recording devices or only one type of fun, mentally stimulating or entertainment activity It also could be used with any currently existing or yet to be developed way to measure and provide a trainee with feedback about their brainwave activity. This subject method is also easily adaptable for use with educational instructional material, such as reading training, psychological tests and brain building exercises by incorporating it into the software that runs these types of programs. Other types of video games or training screens can be modified to use this invention such as standalone or computer integrated media players (e.g., CD, DVD or digital computerized TV electronic device). For example, conventional video or educational game software can be modified to implement this subject system.

The system could alternatively be programmed into an external hardware controller, computer or dedicated purpose device that does not require the modification of the software or media device and would control the positive and negative reinforcement of the motivational activity based on Neurofeedback assessment and training. Further expansion and customization could enable other types of electronic devices, automobiles, airplanes, trucks, or software applications to be accessible only when the threshold criterion has initially been met. For instance, it could be possible to only allow a pilot to fly a plane after he or she has demonstrated that they have the sufficient attentional control required to operate sophisticated mechanical and electronic devices. This subject method can also be adapted to control the launch of productivity applications such as word processing software or web browsers only after desired EEG activity goals have been successfully achieved.

What is claimed is:

1. A method for improving neurofeedback training using a reinforcement system of controlled entertainment-based access, the method comprising:
configuring EEG threshold settings and neurofeedback exercise goals for neurofeedback training;
initiating neurofeedback exercises in a training session;
monitoring a patient engaged in the neurofeedback exercise;
recording EEG data from monitored patient;
determining whether training goals were obtained;
repeating the exercises during a set time limit wherein the goals were not obtained; and
wherein the goals were obtained during the set time period, terminating the exercises wherein the goals were obtained;
permitting access to an entertainment activity for a period of time;
terminating the entertainment activity;
optionally conducting additional neurofeedback training session to provide additional access to the entertainment activity so that a patient is motivated to learn to control EEG psychophysiological functioning in order to improve the patient's mental, emotional and behavioral functioning via access to the entertainment activity.

2. The method of claim 1, wherein the step of determining whether training goals were obtained further comprises the step of controlling the initiation, resumption or pausing of a an independently running cognitive, stimulating or enjoyable activity for the person to view or engage interactively through an electronic device that is simultaneously used to monitor the EEG activity of the patient.

3. A method for improving neurofeedback training using a reinforcement system of controlled entertainment-based access, the method comprising:
configuring EEG threshold settings and neurofeedback exercise goals for neurofeedback training;
monitoring EEG data from a monitored subject;
initiating an entertainment activity for a specified period of time;
conducting neurofeedback training;
checking during the entertainment activity as to whether training time period has expired and
1) terminating neurofeedback training and saving data when time is expired, or
2) continuing neurofeedback training where training time has not expired;
determining whether an EEG training time period has expired wherein the determination that the EEG training time period has not expired comprises repeating the steps of initiating the entertainment activity, conducting neurofeedback training and checking as to whether the entertainment activity time period has expired and wherein the determination that the EEG training time has expired comprises the step of testing whether neurofeedback goals were achieved.

4. The method of claim 3, wherein the step of testing whether neurofeedback goals where obtained during an EEG training time period further comprises the steps of:
saving all neurofeedback data and permitting access to the entertainment activity wherein it is determined that neurofeedback goals were obtained during the EEG training time period; and
wherein it is determined that neurofeedback goals were not obtained, terminating access to the entertainment activity;
conducting additional neurofeedback training exercises until training exercise goals are obtained;
reinitiating entertainment activity when training exercise goals are obtained.
